(19) United States
(12) Patent Application Publication
(10) Pub. No.: US 2012/0203301 A1
(43) Pub. Date: Aug. 9, 2012

(54) METHODS USING TRIGEMINAL NERVE STIMULATION TO TREAT NEUROLOGICAL DISEASES

(75) Inventors: Tracy L. Cameron, Toronto (CA); Peter R. Lando, Dallas, TX (US); Melissa W. Acosta, Fort Worth, TX (US)

(73) Assignee: ADVANCED NEUROMODULATION SYSTEMS, INC., Plano, TX (US)

(21) Appl. No.: 13/339,218

(22) Filed: Dec. 28, 2011

Related U.S. Application Data
(60) Provisional application No. 61/440,157, filed on Feb. 7, 2011.

(51) Int. Cl. A61N 1/372

(52) U.S. Cl. 607/45

(57) ABSTRACT

The present description relates to cranial nerve stimulation to treat a neurological disorder. More particularly, and not by way of limitation, the present invention is directed to a system and method for stimulating a cranial nerve to treat a neurological disorder, such as tinnitus or an anxiety disorder.

### BACKGROUND

The present description relates to cranial nerve stimulation in combination with an event to treat a neurological disorder. More particularly, and not by way of limitation, the present invention is directed to a system and method for stimulating a cranial nerve to treat a neurological disorder, such as tinnitus or an anxiety disorder.

Cranial nerve stimulation has been used successfully to treat a number of nervous system disorders, including epilepsy and other movement disorders, depression and other neuropsychiatric disorders, dementia, coma, migraine headache, obesity, eating disorders, sleep disorders, cardiac disorders (such as congestive heart failure and atrial fibrillation), hypertension, endocrine disorders (such as diabetes and hypoglycemia), and pain, among others. See, e.g., U.S. Pats. Nos. 4,867,164; 5,299,569; 5,269,303; 5,571,150; 5,215,086; 5,188,104; 5,263,480; 6,587,719; 6,609,025; 5,335,657; 6,622,041; 5,916,239; 5,707,400; 5,231,988; and 5,330,515.

Despite the recognition that cranial nerve stimulation may be an appropriate treatment for the foregoing conditions, the fact that detailed neural pathways for many (if not all) cranial nerves remain relatively unknown makes predictions of efficacy for any given disorder difficult. Even if such pathways were known, moreover, the precise stimulation parameters that would energize particular pathways that affect the particular disorder likewise are difficult to predict.

### BRIEF DESCRIPTION OF THE SEVERAL VIEWS OF THE DRAWING

In the following section, the invention will be described with reference to exemplary embodiments illustrated in the figures, in which:

FIG. 1 is illustrative of the trigeminal nerve nucleus in the brainstem and its branches from the trigeminal ganglion and the peripheral branches of the trigeminal nerve.

FIG. 2A and FIG. 2B are illustrative of the dermatomes of the head. FIG. 2A shows the dermatomes areas and FIG. 2B shows the dermatome areas and illustrates several peripheral branches of the trigeminal nerve.

FIG. 3 is illustrative of the nerves on the back of the head, including the occipital area.

FIGS. 4A-4J illustrates example electrical stimulation leads that may be used to electrically stimulate neuronal tissue.

FIG. 5 depicts an exemplary system used to treat a neurological disorder according to one representative embodiment.

FIG. 6 depicts an implantable pulse generator that may be programmed to generate stimulation and an event according to one representative embodiment.

FIG. 7 depicts a representative embodiment for the treatment of tinnitus.

### DETAILED DESCRIPTION

Representative embodiments describe methods to provide stimulation to cranial nerves, peripheral nerves or branches associated with the head and face and/or dermatome areas associated with the head and/or face. The neural stimulation can be provided in combination with an event, for example, an auditory signal can be applied simultaneous or substantially simultaneously while the neural stimulation is occurring to potentially enhance neural plasticity, prevent habituation, or basically enhance efficacy of the treatment protocol or regimen for a neurological disorder.

### I. Stimulation Sites

Cranial nerves are components of the peripheral nervous system that are attached to the brain, rather than the spinal cord. Some cranial nerves relay information from the sense organs to the brain; other cranial nerves control muscles and yet other cranial nerves are part of the autonomic system and relay information to glands or internal organs, such as the heart and lungs.

Cranial nerves that can be stimulated using the methods described herein include, but are not limited to olfactory nerve, optic nerve, oculomoter nerve, trochlear nerve, trigeminal nerve, abducent nerve, facial nerve, vestibulocochlear nerve, glossopharyngeal nerve, vagal nerve, accessory nerve, and the hypoglossal nerve. In addition to cranial nerves, other peripheral nerves that are located in the head and neck may also be stimulated to treat neurological disorders. Such peripheral nerves include, but are not limited to nerves that originate from the spinal nerve roots C2, C3 and C4 that innervate the dermatomes of the head, more specifically, the back of the head and neck. For example, but not limited to the occipital nerve (e.g., greater occipital, lesser occipital, great auricular, posterior division of cervical nerve, cutaneous cervical nerve, and spinal accessory nerve.

A. Trigeminal Nerve

Exemplary embodiments as described herein provide for the treatment of neurological disorders by providing stimulation of the trigeminal nerve and/or branches of the trigeminal nerve.

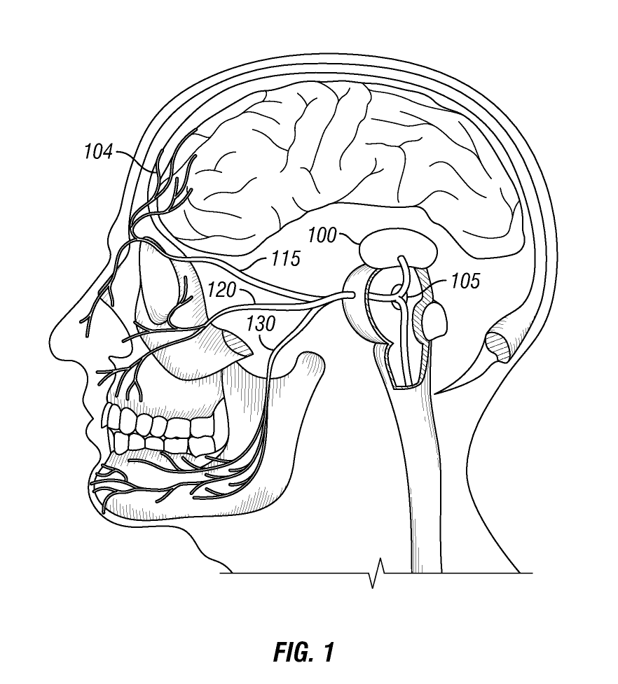

The trigeminal nerve, also known as the fifth cranial nerve, is the largest cranial nerve that has three major divisions, the ophthalmic (V1), maxillary (V2) and mandibular (V3), as shown in FIG. 1. In spite of the trigeminal nerve being primarily a sensory nerve, it also has certain motor functions (biting, chewing, and swallowing).

As shown in FIG. 1, the trigeminal nerve emerges from the brainstem 100 on the midlateral surface of the pons as a large sensory root and a smaller motor root. The sensory ganglion or trigeminal ganglion 110 sits in a depression or the trigeminal cave, also known as Meckle's cave. The trigeminal ganglion is analogous to the dorsal root ganglia of the spinal cord, which contain the cell bodies of incoming sensory fibers from the rest of the body. From the trigeminal ganglion 110, the major divisions of the nerves exit the skull, such as the ophthalmic branch (V1) 115 exits via the superior orbital fissure, the maxillary branch (V2) 120 exits via the foramen rotundum and the mandibular branch (V3) 130 exits via the foramen ovale. The ophthalmic nerve and occasionally the maxillary nerve course through the cavernous sinus before leaving the cranial cavity. As the nerves exit the skull or cranial cavity, each nerve branches extensively 140.

Nucleus of tractus solitarius (NTS) is an integrative center in the brainstem or medulla. Historically, it was considered that the NTS carried and received visceral sensation and taste from the facial (VII), glossopharyngeal (IX) and vagus (X) cranial nerves, however, recent evidence shows a significant number of neurons in the trigeminal nucleus project to the nucleus of tractus solitarius (NTS). Thus, a possibility is that the spinal and trigeminal neurons that project to the NTS may be part of a larger system that integrates somatic and visceral afferent inputs from wide areas of the body. The projections may underlie somatovisceral and/or viscerovisceral reflexes, perhaps with a significant afferent nociceptive component.

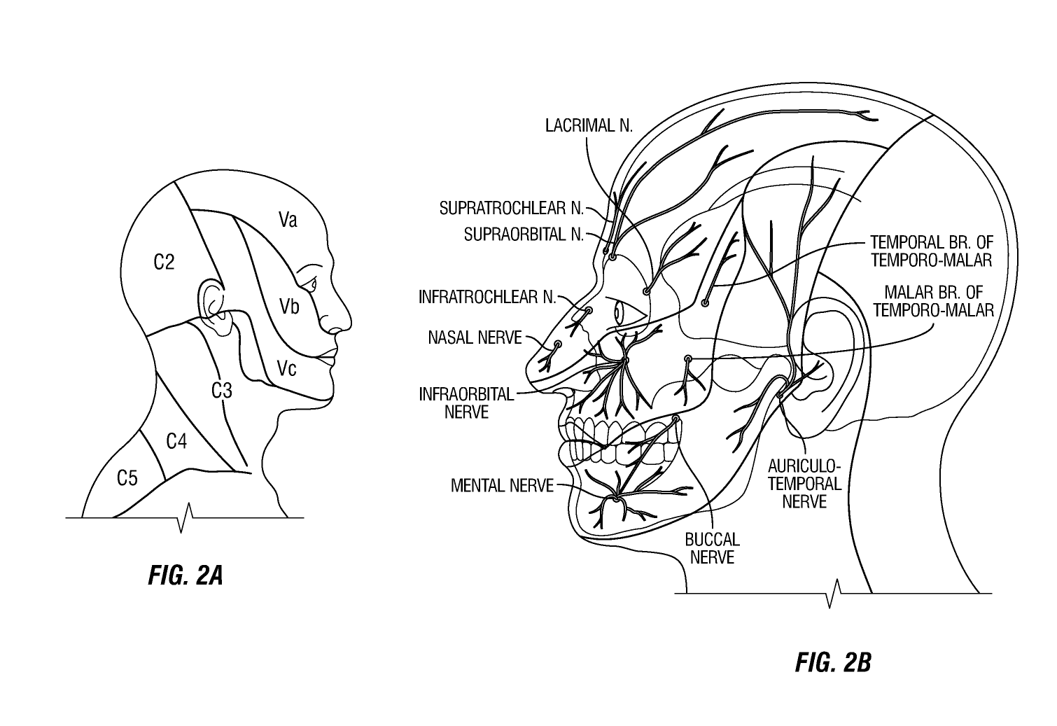

FIG. 2A shows the dermatome areas of the head and face or the sensory areas of the trigeminal nerve or cutaneous innervations of the face. A dermatome area refers to an area of the skin that is innervated by a nerve. The ophthalmic, maxillary and mandibular branches leave the skull through three separate foramina: the superior orbital fissure, the foramen rotundum and the foramen ovale, thereby providing cutaneous innervation to the face: Va, Vb and Vc.

Va is also known as the ophthalmic area because V1 or the ophthalmic nerve branch of the trigeminal nerve and carries sensory information from the scalp and forehead, the upper eyelid, the conjunctiva and cornea of the eye, the nose (including the tip of the nose, except alae nasi), the nasal mucosa, the frontal sinuses, and parts of the meninges (the dura and blood vessels). Peripheral branches of the ophthalmic portion of the trigeminal nerve can be divided into three major division comprising frontal nerve (branches include supratochlear, supraorbital and nerve to frontal sinus), lacrimal nerve and the nasociliary (branches include long and short ciliary, infratochlear, ethmoidal anter (internal nasal and external nasal), and posterior) nerves.

Vb is also known as the maxillary area because it is innervated by the V2 or maxillary nerve branch of the trigeminal nerve and carries sensory information from the lower eyelid and cheek, the nares and upper lip, the upper teeth and gums, the nasal mucosa, the palate and roof of the pharynx, the maxillary, ethmoid and sphenoid sinuses, and parts of the meninges.

Vc is also known as the mandibular area because it is innervated by V3 or the mandibular nerve of the trigeminal nerve and carries sensory information from the lower lip, the lower teeth and gums, the chin and jaw (except the angle of the jaw, which is supplied by C2-C3), parts of the external ear, and parts of the meninges.

FIG. 2B shows the main branches of the trigeminal nerve that exit the foreman and provide cutaneous innervations to the regions of the face. For example, but not limited to, the ophthalamic area has cutaneous innervations from the lacrimal nerve, supratrochlear nerve, supraorbital nerve, the infratrochlear nerve, and the nasal nerve. The maxillary area has cutaneous innervations from, but not limited to, the infraorbital nerve, the malar branch of the temporo-malar nerve, the temoral branch of the temporo-malar nerve. And the mandibular area has cutaneous innervations from, but not limited to, the metnal nerve, and the auriculo-temporal nerve.

B. Other Cranial Nerves

As indicated above, the trigeminal nerve is considered to be a general somatic afferent nerve (GSA) and has neurons that are carried projected to the NTS. In addition to stimulation of the trigeminal nerve, other cranial nerves that are also general somatic afferent nerves and are known to have projections to the NTS can also be stimulated to treat the neurological dysfunctions as described herein. Other cranial nerves, include, but are not limited to the vagal nerve, glossopharyngeal and the facial nerve.

Regarding the vagal nerve, the GSA component of the vagal nerve comprises receptors for pain, temperature, pressure and tactile stimuli that lie in the skin of the back of the ear and the external auditory canal. These neurons synapse on the superior ganglion and enter the lower medulla terminating in the spinal trigeminal nucleus and thus become part of, from a functional perspective, the trigeminal system in which the signals are passed to the cerebral cortex from via projections to the thalamus. Similar to vagal nerve, the glossopharyngeal nerve and the facial nerve also have afferent nerves that synapse within the spinal nucleus of the trigeminal nerve and thus the inputs or information are conveyed to the thalamus and cortex via the trigeminothalamic pathway.

C. Peripheral Nerves

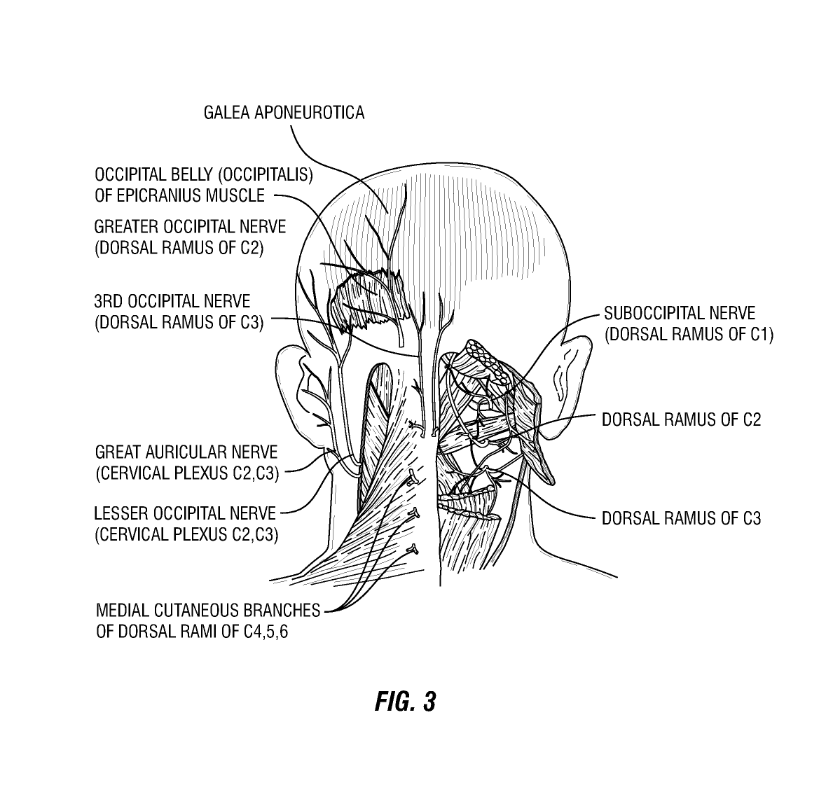

In addition to cranial nerves, it is contemplated that stimulation of peripheral nerves associated with the head and neck can be stimulated to treat the neurological disorders as described herein. Such peripheral nerves include, but are not limited to nerves that originate from the spinal nerve roots C2, C3 and C4 that innervate the dermatomes, as shown in FIG. 2B. For example, but not limited to occipital nerve (e.g., greater occipital, lesser occipital), great auricular, posterior division of cervical nerve, cutaneous cervical nerve, and spinal accessory nerve, as shown in FIG. 3. The C2 dermatome area refers to the area or the dermatome that covers the occiput or occipital area and the top portion of the neck. Yet further, C2 dermatome area includes the neuronal tissue that is located within this area, for example, the C2 dermatome area and its branches innervate the C2 dermatome, as well as any cervical nerves root and/or cranial nerves that may innervate this area.

### II. Stimulation Leads and Devices

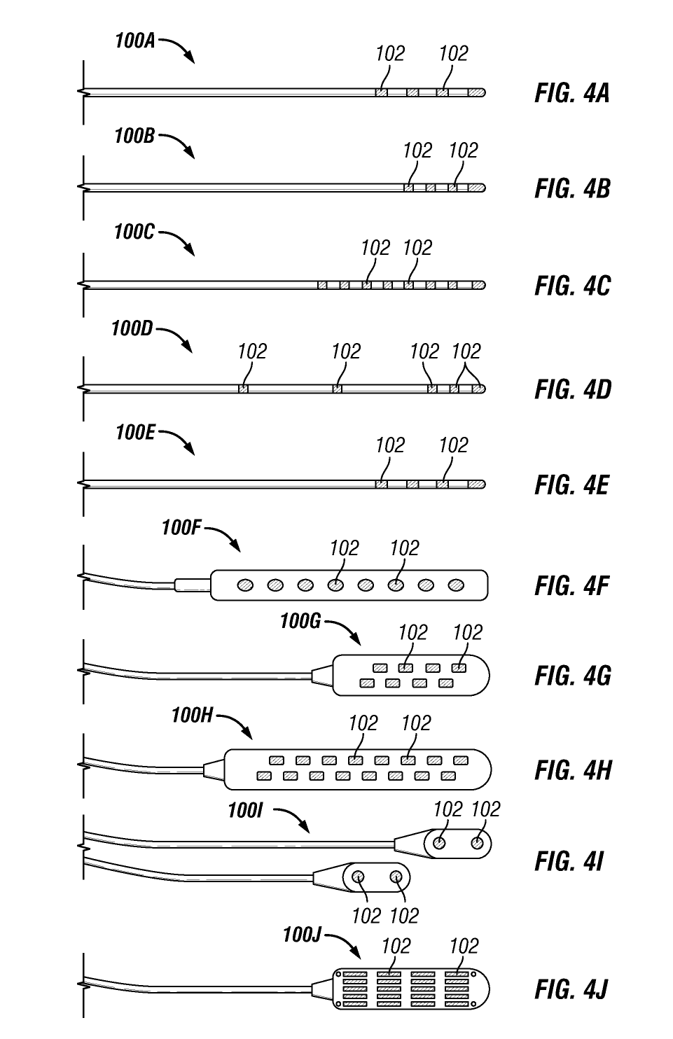

One or more stimulation leads 100, as shown in FIGS. 4A-43 are implanted such that one or more stimulation electrodes 102 of each stimulation lead 100 are positioned or disposed near, adjacent to, directly on or onto, proximate to, directly in or into or within a cranial nerve or other peripheral nerves or dermatome located in the head or face of the patient. The leads shown in FIG. 4 are exemplary of many commercially available leads, such as percutaneous leads, and paddle leads, etc. Examples of commercially available stimulation leads includes a percutaneous OCTRODE® lead or laminotomy or paddle leads or paddle structures such as PENTA® lead or LAMITRODE 44® lead all manufactured by Advanced Neuromodulations Systems. In addition to the illustrated leads, any commercially available nerve cuff or patch electrode or electrode array may also be utilized for stimulation as described herein.

Techniques for implanting stimulation electrodes are well known by those of skill in the art and may be positioned in various body tissues and in contact with various tissue layers; for example, cutaneous, transcutaneous and subcutaneous implantation are employed in some embodiments.

Cranial nerves and other peripheral nerves of the head in which the stimulation leads can be used to stimulate include, but are not limited to, olfactory nerve, optic nerve, oculomoter nerve, trochlear nerve, trigeminal nerve, abducent nerve, facial nerve, vestibulocochlear nerve, glossopharyngeal nerve, vagal nerve, accessory nerve, and hypoglossal nerve. Other peripheral nerves of the head include, but are not limited to the occipital nerve and its branches, the great auricular nerve, the medial cutaneous branches, etc. More particularly, trigeminal nerve or the branches of the trigeminal nerve, for example, but not limited to branches of the ophthalmic division or branch, such as branches of the frontal nerve, lacrimal nerve, or nasociliary nerve, or branches associated in the maxillary area, such as the infraorbital nerve, temporal branch of the temporo-malar nerve, malar branch of the temporor-malar nerve, as shown in FIG. 2B.

One or more stimulation leads 100 can be positioned cutaneously, subcutaneously or transcutaneously such that one or more stimulation electrodes 102 of each stimulation lead 100 are positioned in communication with a branch of the trigeminal nerve as shown in FIG. 1 and FIGS. 2A-2B. It is envisioned that the stimulation lead may be implanted, for example, subcutaneously, or the stimulation may be external, such that the lead is positioned on top of the skin. For example, the stimulation lead is positioned subcutaneously or below the skin but superior or above the cranium or skull. More specifically, the stimulation lead is positioned below the skin and superior to the periostium in the general area above the eye over a branch of the trigeminal nerve. In certain embodiments, the stimulation lead is positioned supraorbital below the skin and superior to the periostium. Yet further, the lead may also be placed in an infraorbital position, for example, the stimulation lead can be positioned below the skin and superior to the periostium in the general area by the nose such that the lead stimulates the infraorbital branch or the nasal branch of the trigeminal nerve. Depending upon the treatment regimen, the stimulation lead can be placed unilaterally or bilaterally.

In certain embodiments one or more stimulation electrodes are positioned in the C2 dermatome area, subcutaneously, cutaneously, or transcutanelously. For subcutaneous, the electrodes are positioned superior to the galea. Within certain areas of the C2 dermatome area, there is little or no muscle, this area primarily consists of fat, fascia, perostium, and neurovascular structures (e.g., galea), as shown in FIG. 3. Thus, the advantage implanting a stimulation lead in this area is that there will be no to little muscular contraction. One of skill in the art is aware that stimulation of the C2 dermatome area may result in stimulation of various neuronal structures, for example, but not limited to the C2 dermatome area, C3 dermatome, cranial nerves or other cervical nerves located in this general area. More specifically, the electrode can be implanted in a subcutaneous fashion such that the electrode is positioned below the skin, above the bone on the back of the head or superior to the periosteum. On the back of the head, the probe is positioned in the C2 dermatome area or positioned at the back of the patient's head at about the level of the ear.

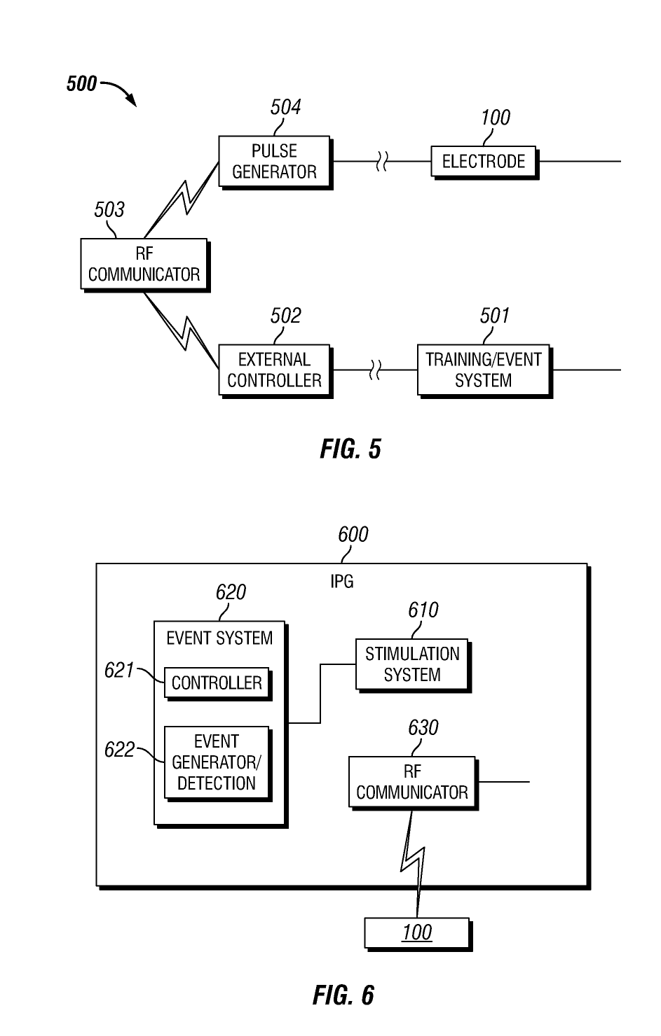

FIG. 5 depicts a stimulation system 500 for stimulating neural tissue of a patient and providing a training/event system. Stimulation system 500 comprises a stimulation source or pulse generator 504 which is electrically coupled (possibly through extension leads) to stimulation lead 100, for example, those shown in FIG. 4A-43 or any other commercially available stimulation lead or electrode array. Still further, stimulation system 500 may be an implantable programming device (IPG) or may comprise a device that is located external to the body of the patient or transcutaneous. In “transcutaneous” electrical nerve stimulation (TENS) the stimulation source is external to the patient's body, and may be worn in an appropriate fanny pack or belt, and the stimulation lead is in communication with the stimulation source, either remotely or directly, the stimulation lead can also be external, for example a patch type electrode that is placed on top of the skin, or a patch having an electrode array attached to the skin. In certain embodiments, a patch electrode may be placed or positioned above the eye, near the nose, on the forehead or back of the head such that it stimulates the desired nerves as illustrated in FIGS. 2A, 2B and 3.

Stimulation system 500 also provides an external controller 502 that is capable of controlling the training/event system 501. The pulse generator 504 and external controller 502 are communicably connected either through a direct connection (not shown) or via a wireless connection such as utilizing a separate radio frequency transmitter/receiver 503. It is contemplated that each of pulse generator 504 and external controller 502 could each include the necessary transmitter/receiver components to communicate directly therebetween. The training/event system 501 can receive instruction from the external controller 502 or it is possible that it can also provide instruction to the external controller 502. The training/event system 501 generates a desired event depending upon the disease state to be treated. The generated event by training/event system 501 is paired in combination with the electrical stimulation produced by pulse generator 504 to electrode 100 to induce plasticity of the brain.

The external controller 502 or training/event system 501 generally provides the nature of the pairing of the event and the stimulation. The stimulation and the event or training can occur in a manner that is simultaneous, substantially simultaneous, coexisting, concurrent, or synchronous. That is, the stimulation and the event or training can occur at the same time and/or such that there is at least some overlap in the timing. In other embodiments, the nature of the event and stimulation are such that they are sequential, asynchronous, separate, following or preceding one another, for example, the stimulation may lead the start of the training while in other examples the stimulation may follow the start of the training. In some cases, the stimulation is shorter in duration than the training, such that the stimulation occurs near the beginning of the training.

FIG. 5 shows a device in which the pulse generator 504 is physically separate from the external controller 502 or event/training controller 501. These devices can be physically separate with information being communicated via any standard means, such as radio frequency. Another embodiment employs a pulse generator 600 that is capable of performing a number of operations, as shown in FIG. 6. Such operations include, but are not limited to a stimulation system 610 which comprises pulse generating circuitry and any other components required to generate stimulation pulses; an event system 620 that comprises a controller 621 and event generator/detection system 622; an RF communicator 630 to relay information from the pulse generator 600 to the event device, etc. An example of pulse generating circuitry is described in U.S. Patent Publication No. 20060170486 entitled “PULSE GENERATOR HAVING AN EFFICIENT FRACTIONAL VOLTAGE CONVERTER AND METHOD OF USE,” which is incorporated herein by reference. A microprocessor and associated charge control circuitry for an implantable pulse generator is described in U.S. Patent Publication No. 20060259098, entitled “SYSTEMS AND METHODS FOR USE IN PULSE GENERATION,” which is incorporated herein by reference. Circuitry for recharging a rechargeable battery of an implantable pulse generator using inductive coupling with an external charging device is described in U.S. patent Ser. No. 11/109,114, entitled “IMPLANTABLE DEVICE AND SYSTEM FOR WIRELESS COMMUNICATION,” which is incorporated herein by reference.

The training/event system 501 or event device 640 can provide sensory information (such as visual, auditory, olfactory, tactile or any other suitable sensory information); motor information (such as motor training with or without robotic assistance, motor training with human assistance, such as a healthcare professional passively moving the body part); cognitive or emotional information (such as psychotherapy, showing images, memory/event recall, etc.); chemical events, etc.

Exemplary devices that can be utilized to generate an event include any devices that are capable of generating such information, for example, auditory information or audible devices that can be used include any device that is capable of generating and audible sound, such as a simple tone, a complex tone, etc. Such devices include, earphones, ear buds, headphones, hearing aides, speakers, other types of machines or equipment that generate an audible sound.

Other devices that can trigger sensory information can include a visual system (such as any type of monitor that relays images, colors, figures, etc.); olfactory systems (such as any system that relays smells, i.e., masks, breathing machines, fragrances, atomizers, etc); tactile systems (such as pricking, touching, etc.).

It is envisioned that any event system can be in communication with the controller via any known or standard communication means, for example, the communication can be established using wires or can be established wirelessly, such using radio frequency. Such radio frequency devices that can be employed include Bluetooth technology.

With reference to FIG. 6, in certain instances, the event system 620 may comprise an event generator/detector 622, such that an event can be detected and stimulation is applied or initiated based upon the detection of the event. Thus, the detector can monitor external or internal events, including heart-rate, blood pressure, temperature, chemical levels or any other parameter that may indicate an event. Other physiological responses that can be detected or monitored include changes that can be recorded using functional imaging, for example, PET, MEG, EEG, fMRI, changes in local pH, ionic concentrations (i.e., potassium), concentration of neurotransmitters, changes in temperature, and changes in metabolic rate markers, etc.

In alternative embodiments as described herein, the timing of the training or event and/or the stimulation may be controlled manually. Further therapies and training may include training triggered timing or physical condition feedback to provide a closed-loop system.

In another embodiment, the stimulation is percutaneous. In “percutaneous” electrical nerve stimulation (PENS), needles are inserted to an appropriate depth around or immediately adjacent to a stimulation site, and then stimulated.

In addition to electrical stimulation, it may be desirable to use a drug delivery system in combination with the event system as described above. Drug delivery may be used independent of or in combination with a lead/electrode to provide electrical stimulation and chemical stimulation. When used, the drug delivery catheter is implanted such that the proximal end of the catheter is coupled to a pump and a discharge portion for infusing a dosage of a pharmaceutical or drug. Implantation of the catheter can be achieved using similar techniques as discussed above for implantation of electrical leads, which is incorporated herein. The distal portion of the catheter can have multiple orifices to maximize delivery of the pharmaceutical while minimizing mechanical occlusion. The proximal portion of the catheter can be connected directly to a pump or via a metal, plastic, or other hollow connector, to an extending catheter.

Any type of infusion pump can be used, for example, “active pumping” devices or so-called peristaltic pumps are described in U.S. Pat. Nos. 4,692,147, 5,840,069, and 6,036,459, which are incorporated herein by reference in their entirety. Peristaltic pumps are used to provide a metered amount of a drug in response to an electronic pulse generated by control circuitry associated within the device. An example of a commercially available peristaltic pump is SynchroMed® implantable pump from Medtronic, Inc., Minneapolis, Minn.

Other pumps that may be used include accumulator-type pumps, for example certain external infusion pumps from Minimed, Inc., Northridge, Calif. and Infusaid® implantable pump from Strato/Infusaid, Inc., Norwood, Mass. Passive pumping mechanisms can be used to release an agent in a constant flow or intermittently or in a bolus release. Passive type pumps include, for example, but are not limited to gas-driven pumps described in U.S. Pat. Nos. 3,731,681 and 3,951,147; and drive-spring diaphragm pumps described in U.S. Pat. Nos. 4,772,263, 6,666,845, 6,620,151 all of which are incorporated by reference in their entirety. Pumps of this type are commercially available, for example, Model 3000® from Arrow International, Reading, Pa. and IsoMed® from Medtronic, Inc., Minneapolis, Minn.; AccuRx® pump from Advanced Neuromodulation Systems, Inc., Plano, Tex.

In certain embodiments, the catheter can be in the form of a lead catheter combination, similar to the ones described in U.S. Pat. No. 6,176,242 and U.S. Pat. No. 5,423,877, which are incorporated herein by reference in their entirety.

A chemical stimulation or drug delivery system can comprises a system to control release of neurotransmitters (e.g., glutamate, acetylcholine, norepinephrine, epinephrine, dopamine), chemicals (e.g., zinc, magnesium, lithium) and/or pharmaceuticals that are known to alter the activity of nerves or neuronal tissue. For example, infusion formulation delivery system can utilize a control system having an input-response relationship. A sensor or detector generates a sensor signal representative of a system parameter input (such as levels of neurotransmitters), and provides the sensor signal to a controller. The controller receives the sensor signal and generates commands that are communicated to the infusion formulation delivery device. The infusion formulation delivery device then delivers the infusion formulation output to stimulation site or target site at a determined rate and amount in order to control the system parameter.

The sensor may comprise a sensor, sensor electrical components for providing power to the sensor and generating the sensor signal, a sensor communication system for carrying the sensor signal to controller, and a sensor housing for enclosing the electrical components and the communication system. Controller may include one or more programmable processors, logic circuits, or other hardware, firmware or software components configured for implementing the control functions described herein, a controller communication system for receiving the sensor signal from the sensor, and a controller housing for enclosing the controller communication system and the one or more programmable processors, logic circuits, or other hardware, firmware or software components. The infusion formulation delivery device may include a suitable infusion pump, infusion pump electrical components for powering and activating the infusion pump, an infusion pump communication system for receiving commands from the controller, and an infusion pump housing for enclosing the infusion pump, infusion pump electrical components, and infusion pump communication system. Such systems are described in U.S. Pat. No. 6,740,072, which is incorporated herein by reference in its entirety.

Herein, stimulating drugs comprise medications, anesthetic agents, synthetic or natural peptides or hormones, neurotransmitters, cytokines and other intracellular and intercellular chemical signals and messengers, other agents such as zinc and the like. In addition, certain neurotransmitters, hormones, and other drugs are excitatory for some tissues, yet are inhibitory to other tissues. Therefore, where, herein, a drug is referred to as an “excitatory” drug, this means that the drug is acting in an excitatory manner, although it may act in an inhibitory manner in other circumstances and/or locations. Similarly, where an “inhibitory” drug is mentioned, this drug is acting in an inhibitory manner, although in other circumstances and/or locations, it may be an “excitatory” drug. In addition, stimulation of an area herein includes stimulation of cell bodies and axons in the area.

Similarly, excitatory neurotransmitter agonists (e.g., norepinephrine, epinephrine, glutamate, acetylcholine, serotonin, dopamine), agonists thereof, and agents that act to increase levels of an excitatory neurotransmitter(s) (e.g., edrophonium; Mestinon; trazodone; SSRIs (e.g., flouxetine, paroxetine, sertraline, citalopram and fluvoxamine); tricyclic antidepressants (e.g., imipramine, amitriptyline, doxepin, desipramine, trimipramine and nortriptyline), monoamine oxidase inhibitors (e.g., phenelzine, tranylcypromine, isocarboxasid)), generally have an excitatory effect on neural tissue, while inhibitory neurotransmitters (e.g., dopamine, glycine, and gamma-aminobutyric acid (GABA)), agonists thereof, and agents that act to increase levels of an inhibitory neurotransmitter(s) generally have an inhibitory effect (e.g., benzodiasepine (e.g., chlordiazepoxide, clonazepam, diazepam, lorazepam, oxazepam, prazepam alprazolam); flurazepam, temazepam, or triazolam). (Dopamine acts as an excitatory neurotransmitter in some locations and circumstances, and as an inhibitory neurotransmitter in other locations and circumstances.) However, antagonists of inhibitory neurotransmitters (e.g., bicuculline) and agents that act to decrease levels of an inhibitory neurotransmitter(s) have been demonstrated to excite neural tissue, leading to increased neural activity. Similarly, excitatory neurotransmitter antagonists (e.g., prazosin, and metoprolol) and agents that decrease levels of excitatory neurotransmitters may inhibit neural activity. Yet further, lithium salts, anesthetics (e.g., lidocane), and magnesium may also be used in combination with electrical stimulation.

In addition to electrical stimulation and/or chemical stimulation, other forms of stimulation can be used, for example magnetic, or thermal or combinations thereof. Magnetic stimulation can be provided by internally implanted probes or by externally applied directed magnetic fields, for example, U.S. Pat. Nos. 6,592,509; 6,132,361; 5,752,911; and 6,425,852, each of which is incorporated herein in its entirety. Quick pulses of magnetic stimulation can be applied externally or transcranially, for example repetitive transcranially magnetic stimulation (rTMS). Thermal stimulation can be provided by using implanted probes that are regulated for heat and/or cold temperatures which can stimulate or inhibit neuronal activity, for example, U.S. Pat. No. 6,567,696, which is incorporated herein by reference in its entirety.

It is envisaged that the patient will require intermittent assessment with regard to patterns of stimulation. Different electrodes on the lead can be selected by suitable computer programming, such as that described in U.S. Pat. No. 5,938,690, which is incorporated by reference here in full. Utilizing such a program allows an optimal stimulation pattern to be obtained at minimal voltages. This ensures a longer battery life for the implanted systems.

In certain embodiments, the stimulation may be continuous or administered as needed. In other embodiment, the stimulation is provided in a burst-type pattern in order to modulate effects such as nerve plasticity or habitation. It is known that some neurons fire in packets of action potentials followed by periods of quiescence (bursts) while others, within the same stage of sensory processing, fire in a tonic manner. Thus, burst and tonic firing might be processing information in parallel in certain sensory systems (Ozwald et al., 2004; Chacron et al. 2004), and thus, in order to enhance neural plasticity or prevent habitation, burst type stimulation can be applied in combination with an event/training system as described herein.

Burst-type stimulation can be achieved using a conventional neuromodulation device as described herein that is adapted to apply burst stimulation to nerve tissue of a patient by modifying the software instructions and/or stimulation parameters stored in the devices. Specifically, conventional neuromodulation devices typically include a microprocessor and a pulse generation module. The pulse generation module generates the electrical pulses according to a defined pulse width and pulse amplitude and applies the electrical pulses to defined electrodes. The microprocessor controls the operations of the pulse generation module according to software instructions stored in the device and accompanying stimulation parameters. An example of a commercially available neuromodulation device that can be modified or programmed to apply burst stimulation includes the EON®, manufactured by Advanced Neuromodulation Systems, Inc.

These conventional neuromodulation devices can be adapted by programming the microprocessor to deliver a number of spikes (relatively short pulse width pulses) that are separated by an appropriate inter-spike interval. Thereafter, the programming of the microprocessor causes the pulse generation module to cease pulse generation operations for an inter-burst interval. The programming of the microprocessor also causes a repetition of the spike generation and cessation of operations for a predetermined number of times. After the predetermined numbers of repetitions have been completed, the microprocessor can cause burst stimulation to cease for an amount of time and resume thereafter.

The microprocessor can be programmed to allow the various characteristics of the burst stimulus to be set by a health care professional to allow the burst stimulus to be optimized to treat the neurological disorder. For example, the spike amplitude, the inter-spike interval, the inter-burst interval, the number of bursts to be repeated in succession, the amplitude of the various pulses, and other such characteristics could be controlled using respective parameters accessed by the microprocessor during burst stimulus operations. These parameters could be set to desired values by an external programming device via wireless communication with the implantable neuromodulation device.

In another embodiment, a neuromodulation device can be implemented to apply burst stimulation using a digital signal processor and one or several digital-to-analog converters. The burst stimulus waveform could be defined in memory and applied to the digital-to-analog converter(s) for application through electrodes of the medical lead. The digital signal processor could scale the various portions of the waveform in amplitude and within the time domain (e.g., for the various intervals) according to the various burst parameters.

Examples of burst stimulation are found in U.S. Published Application No. US20060095088, and incorporated herein by reference in its entirety. The burst stimulation may generate bursts of a plurality of electrical pulses with an inter-burst frequency in the range of about 1 Hz to about 100 Hz, more particular, in the range of about 1 Hz to about 50 Hz, and more particularly, about 40 Hz. The inter-burst interval has a duration in the range of about 1 milliseconds to about 5 seconds, more preferably, about 10 milliseconds to about 300 milliseconds. The inter-burst interval need not be constant and can be varied in a programmable manner or varied pseudo-randomly by the pulse generator (e.g., random or irregular harmonics).

### III. Treatment of Disorder

Using the above described stimulation system, the nerve or nervous tissue or area in the head or face as shown in FIG. 2A, 2B or 3 is stimulated in an effective amount or effective treatment regimen to decrease, reduce, modulate or abrogate the neurological disorder or condition. Such treatment regimes may require neural stimulation in combination with an event.

A. Patient Selection

Patients to be treated according to some representative embodiments can be selected, identified and/or diagnosed based upon the accumulation of physical, chemical, and historical behavioral data on each patient. One of skill in the art is able to perform the appropriate examinations to accumulate such data. One type of examination can include neurological examinations, which can include mental status evaluations, which can further include a psychiatric assessment. Other types of assessments for movement disorders may include such assessments for example using the Unified Parkinson's Disease Rating Scale (UPDRS). Still further, other types of examinations can include, but are not limited to, motor examination, cranial nerve examination, cognitive assessment and neuropsychological tests (i.e., Minnesota Multiphasic Personality Inventory, Beck Depression Inventory, or Hamilton Rating Scale for Depression). Other types of assessment for tinnitus, for example, can include but are not limited to Visual Analogue Scales (VAS) and Tinnitus Handicap Inventory (THI). In addition to neurological testing, routine hematological and/or biochemistry testing may also be performed.

In addition to the above examinations, imaging techniques can be used to determine normal and abnormal brain function that can result in disorders. Thus, once the patient is identified from the above clinical examinations, imaging techniques can be further utilized to provide the region of interest in which the electrodes are to be implanted. Functional brain imaging allows for localization of specific normal and abnormal functioning of the nervous system. This includes electrical methods such as electroencephalography (EEG), magnetoencephalography (MEG), single photon emission computed tomography (SPECT), as well as metabolic and blood flow studies such as functional magnetic resonance imaging (fMRI), and positron emission tomography (PET) which can be utilized to localize brain function and dysfunction.

B. Treatment Protocols

Certain neurological disorders that may be treated according to the methods described herein may include, but are not limited to attention or cognitive disorders (e.g., Autistic Spectrum Disorders); mood disorder (e.g., major depressive disorder, bipolar disorder, and dysthymic disorder) or an anxiety disorder (e.g., panic disorder, posttraumatic stress disorder, obsessive-compulsive disorder and phobic disorder); neurodegenerative diseases (e.g., multiple sclerosis, Alzheimer's disease, amyotrophic lateral sclerosis (ALS), Parkinson's disease, Huntington's Disease, Guillain-Barre syndrome, myasthenia gravis, and chronic idiopathic demyelinating disease (CID)), movement disorders (e.g, dyskinesia, tremor, dystonia, chorea and ballism, tic syndromes, Tourette's Syndrome, myoclonus, drug-induced movement disorders, Wilson's Disease, Paroxysmal Dyskinesias, Stiff Man Syndrome and Akinetic-Ridgid Syndromes and Parkinsonism), epilepsy, tinnitus, pain, phantom pain, diabetes neuropathy, one skilled in the art appreciates that the invention may also find application in conjunction with enhancing or diminishing any neurological or psychiatric function, not just an abnormality or disorder. Neurological activity that may be modulated utilizing the stimulation protocol described herein can include, but not be limited to, normal functions such as alertness, conscious state, drive, fear, anger, anxiety, repetitive behavior, impulses, urges, obsessions, euphoria, sadness, and the fight or flight response, as well as instability, vertigo, dizziness, fatigue, photofobia, concentration dysfunction, memory disorders, headache, dizziness, irritability, fatigue, visual disturbances, sensitivity to noise (misophonia, hyperacusis, phonofobia), judgment problems, depression, symptoms of traumatic brain injury (whether physical, emotional, social or chemical), autonomic functions, which includes sympathetic and/or parasympathetic functions (e.g., control of heart rate), somatic functions, and/or enteric functions.

A patient or patient is administered a therapeutically effective stimulation so that the patient has an improvement in the parameters relating to the neurological disorder or condition including subjective measures such as, for example, neurological examinations and neuropsychological tests, motor examination, visual analog scale (VAS) and cranial nerve examination, and objective measures including use of additional psychiatric medications, such as anti-depressants, or other alterations in cerebral blood flow or metabolism and/or neurochemistry.

Patient outcomes may also be tested by health-related quality of life (HRQL) measures: Patient outcome measures that extend beyond traditional measures of mortality and morbidity, to include such dimensions as physiology, function, social activity, cognition, emotion, sleep and rest, energy and vitality, health perception, normal eating habits or behaviors (i.e., regained appetite or reduced appetite) and general life satisfaction. (Some of these are also known as health status, functional status, or quality of life measures.)

Treatment regimens may vary as well, and often depend on the health and age of the patient. Obviously, certain types of disease will require more aggressive treatment, while at the same time, certain patients cannot tolerate more taxing regimens. The clinician will be best suited to make such decisions based on the known patient's history.

For purposes of this application, beneficial or desired clinical results include, but are not limited to, alleviation of symptoms, improvement of symptoms, diminishment of extent of disease, stabilized (i.e., not worsening) state of disease, delay or slowing of disease progression, amelioration or palliation of the disease state, and remission (whether partial or total), whether objective or subjective. The improvement is any observable or measurable improvement. Thus, one of skill in the art realizes that a treatment may improve the patient condition, but may not be a complete cure of the disease.

1. Tinnitus Treatment Protocols

Tinnitus is a noise in the ears, often described as ringing, buzzing, roaring, or clicking. Subjective and objective forms of tinnitus exist, with objective tinnitus often caused by muscle contractions or other internal noise sources in the area proximal to auditory structures. In certain cases, external observers can hear the sound generated by the internal source of objective tinnitus. In subjective forms, tinnitus is audible only to the patient. Tinnitus varies in perceived amplitude, with some patients reporting barely audible forms and others essentially deaf to external sounds and/or incapacitated by the intensity of the perceived noise.

The auditory system consists of two main parallel pathways supplying auditory information to the cerebral cortex: the topographically organized lemniscal (classical) system, and the non-topographic extralemniscal (non-classical) system. The classical pathways use the ventral thalamus, the neurons of which project to the primary auditory cortex whereas the non-classical pathways use the medial and dorsal thalamic nuclei that project to the secondary auditory cortex and association cortices, thus bypassing the primary cortex (Møller, 2003). While neurons in the classical pathways only respond to one modality of sensory stimulation, many neurons in the non-classical pathway respond to more than one modality. Neurons in the ventral thalamus fire in a tonic or semi-tonic mode while neurons in the medial and dorsal thalamus fire in bursts (He and Hu, 2002; Hu et al., 1994). The non-classical pathways receive their input from the classical pathways, which means that the ascending auditory pathways are a complex system of at least two main parallel systems that provide different kinds of processing and which interact with each other in a complex way. Both systems provide sensory input to the amygdala through a long cortical route, and in addition, the non-classical pathways provide subcortical connections to the lateral nucleus of the amygdala from dorsal thalamic nuclei (LeDoux, 1993).

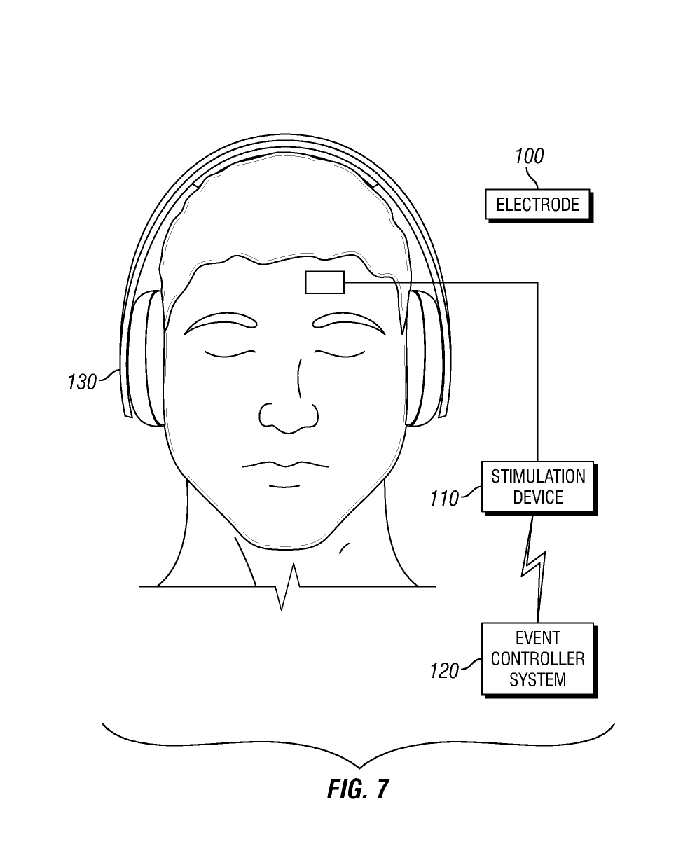

More specifically, regarding the treatment of tinnitus. A patient is administered stimulation in combination with a timed event. An exemplary embodiment is shown in FIG. 7. For example, the stimulation lead 100 is positioned such at least one electrode of the stimulation is in communication with a branch of the trigeminal nerve. The stimulation lead may be placed externally, such as on top of the skin of the forehead or the stimulation lead may be implanted internally such that at least one electrode is positioned subcutaneously in the general area of a branch of the trigeminal nerve or in the general area of a dermatome. As described herein above, the stimulation device 110 comprises a pulse generator that may be implantable or may be external. The event controller system 120 can be a physically separated system that communicates with the stimulation device via any known conventional techniques or the event controller or system 120 can be incorporated into the stimulation device as described in FIG. 6. FIG. 7 further shows that the event is delivered via headphones 130. It is envisioned that virtually any instrument capable of delivering audible sounds to the patient can be utilized in this treatment regimen. The headphones can be remotely or wirelessly capable of communicating with the event controller or system 120 or it is possible that the headphones may be capable of wirelessly communicating with the stimulation device 110 directly.

Tinnitus treatments, for example, may consist of brief audible sounds including selected therapeutic frequencies combined or timed with neural stimulations. Since the duration of the sounds may vary, precision of the stimulation with the sounds or training may be controlled more precisely with a computer, for example, an external programming device that can be utilized by a health professional to initiate or control the timing or the audible signals. The external programming system may include a processor-based computing device, such as a computer, a personal digital assistant (PDA) device or other suitable computing devices.

The neural stimulation that is applied in combination with the audible sounds can be such that it is delivered in a tonic mode or a burst-type pattern to alter activities of either the topographically organized lemniscal (classical) system or the non-topographic extralemniscal (non-classical) system. Yet further, tinnitus cases are commonly complex in that the patient suffers from more than one type (i.e. pure tone, narrow band, white noise) of tinnitus in one or both ears. As such, it may be necessary to apply a combination of stimulation (tonic stimulation and burst type stimulation) parameters with the event/training to alleviate the symptoms.

An improvement in the patient's tinnitus may be measured using parameters relating to tinnitus including informal questioning of the patient, formal subjective testing and analysis according to one or more audiology test, for example the Goebel tinnitus questionnaire or other validated tinnitus questionnaires, audiometry, tinnitus matching, impedence, BAEP, and OAE. The improvement is any observable or measurable improvement. Thus, one of skill in the art realizes that a treatment may improve the patient condition, but may not be a complete cure of the disease.

2. Mood and/or Anxiety-Related Treatment Protocols

In the treatment of mood or anxiety-related disorders, such as post-traumatic stress, obsessive-compulsive disorder, depression, addiction or other anxiety related disorders, exposure or extinction therapy or psychotherapy may be used in combination with the stimulation systems described herein.

As described previously, an event can be administered or timed with neural stimulation. The event that can be used in combination with the neural stimulation can include a variety of types of events that provoke the patient's mood or anxiety-related disorder. For example, the event can be an emotional event, such as, using emotional stimuli that can include, but are not limited to disturbing stimuli, sad stimuli, neutral stimuli, happy stimuli, exhilarating stimuli, etc. Those of skill in the art are cognizant that such stimuli can be presented to the patient in a form of images from the International Affective Picture Series set (Lang et al. 1988). Other emotional stimuli can include a mood induction task, for example, a sad recount of autobiographical nature or reading, listening, or writing to induce a mood response. Still further, visual stimuli, such as art work can also be used to induce a mood or emotional response.

In addition to triggering an emotional event, conditioning training can be used to induce an obsessive-compulsive (OCD) trait, for example, touching something dirty can induce a cleanliness OCD trait. Other stimuli to induce memory or cognition can be used, for example, Wechsler Adult Intelligence Scale-Third Edition (WMS-III), Wechsler Memory Scale-Third Edition (WMS-III), Rey Auditory Verbal Learning Test (RAVLT), California Verbal Learning Test (CVLT), Rey-Osterrieth Complex Figure, or Mini-Mental State Exam (MMSE).

It is readily apparent to one skilled in the art that various embodiments and modifications can be made to the representative embodiments in this application without departing from the scope of the appended claims.

All patents and publications mentioned in the specifications are indicative of the levels of those skilled in the art to which the invention pertains. All patents and publications are herein incorporated by reference to the same extent as if each individual publication was specifically and individually indicated to be incorporated by reference

What is claimed:

1. A method for reducing tinnitus, comprising:
using a stimulation source to generate electrical stimulation energy that is delivered to the target tissue via electrodes of an electrical stimulation lead, wherein the electrodes are positioned below the patient's skin and superior to the skull such that the electrodes are over a branch of the trigeminal nerve;
using an event generator providing an audible sound; and
using a controller that is communicable connected to the stimulation source and the event generator, wherein the controller provides instructions to the stimulation source to deliver electrical stimulation to the nerve during an event generated by the event generator.

2. The method of claim 1, wherein the electrical stimulation and the event occur substantially simultaneously.

3. The method of claim 1, wherein the branch of the trigeminal nerve comprises a branch in the ophthalmic area.

4. The method of claim 1, wherein the branch of the trigeminal nerve comprises a branch in the maxillary area.
